(19) United States
(12) Patent Application Publication
(10) Pub. No.: US 2008/0214599 A1
(43) Pub. Date: Sep. 4, 2008

(54) Use of Par-1/Par-4 Inhibitors for Treating or Preventing Vascular Diseases

(76) Inventors: John Thomas Brandt, Indianapolis, IN (US); Victor Leonid Serebruany, West Friendship, MD (US); Kenneth John Winters, Memphis, TN (US)

Correspondence Address: ELI LILLY & COMPANY, PATENT DIVISION, P.O. BOX 6288, INDIANAPOLIS, IN, 46206-6288, US

(21) Appl. No.: 11/996,380

(22) PCT Filed: Aug. 8, 2006
(86) PCT No.: PCT/US06/30831

§ 371 Date: Jan. 22, 2008

Related U.S. Application Data
(60) Provisional application No. 60/709,937, filed on Aug. 19, 2005.

(51) Int. Cl. A61K 31/4365; A61P 3/00; A61P 9/00

(52) U.S. Cl. 514/301

(57) ABSTRACT

Use of a compound of formula (I) or a compound selected from the group consisting of a prasugrel metabolite, clopidogrel, a clopidogrel metabolite, ticlopidine, a ticlopidine metabolite, cangrelor, a cangrelor metabolite, AZD-6140, and an AZD-6140 metabolite for the treatment of and/or prevention of coagulation induced vascular diseases and recurrence thereof in a patient in need thereof.

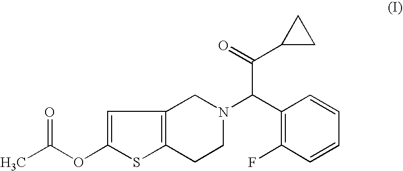

### FIELD OF THE INVENTION

This invention relates to a novel method for the treatment of vascular diseases related to inhibition of the G coupled Protease Activating Receptors 1/4 (PAR-1/PAR-4) receptor expression.

### BACKGROUND OF THE INVENTION

Vascular Disease including myocardial infarction and ischemic stroke is a leading cause of death and disability. While the processes causing vascular disease(s) are complex and not completely understood, an underlying etiology common to the numerous theories includes atherosclerosis due to atherosclerotic lesion formation and the disruption of plaques leading to thrombosis or thromboembolism.

The platelet aggregation inhibitor clopidogrel (marketed as Plavix® and Iscover®) has achieved relative success in reducing the untoward effects of cardiovascular diseases caused by or exacerbated by platelet activation and/or aggregation. Platelet aggregation inhibition and the prevention of embolism is only one of the possible points of intervention to prevent or minimize the incidence of vascular diseases. Thus, there is a need to discover and develop pharmaceutical agents which are effective at treating or preventing vascular diseases caused by or exacerbated by different or multiple sites in the cascade of events leading to various vascular events.

U.S. Pat. No. 5,288,726, discloses the preparation and use of compounds useful for the treatment and/or prevention of thrombosis and thromboembolism. U.S. Pat. No. 6,693,115 discloses acid addition salts (notably the compound CS-747.HCl (now Prasugrel) particularly useful and beneficial for the treatment and/or prevention of thrombosis and thromboembolism.

U.S. Pat. Nos. 4,529,596, 4,847,265, and 6,429,210B1 disclose compounds, pharmaceutical compositions, salts, enantiomers and/or polymorphs of clopidogrel useful for the treatment of thrombosis via inhibition of platelet aggregation.

The above disclosures provide compounds, methods and/or pharmaceutical compositions for the treatment of thrombosis and/or thromboembolism. Nevertheless, there remains a need to discover and develop pharmaceutical agents that are effective at treating or preventing coagulation induced vascular diseases caused by or exacerbated by the cascade of events related to PAR-1/PAR-4 thrombin receptor activity leading to various vascular events.

### SUMMARY OF THE INVENTION

The present invention relates to the use of a compound of formula I

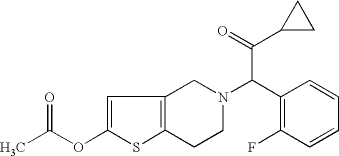

as a PAR-1/PAR-4 thrombin receptor inhibitor for the treatment and/or prevention of coagulation induced vascular diseases and recurrence thereof, in a patient in need thereof.

The present invention relates to the use of a compound selected from the group consisting of clopidogrel, a clopidogrel metabolite, ticlopidine, a ticlopidine metabolite, cangrelor, a cangrelor metabolite, AZD-6140, and an AZD-6140 metabolite for the treatment and/or prevention of coagulation induced vascular diseases and recurrence in a patient in need thereof.

The present invention relates to a method of reducing platelet activation and thrombin generation state of an individual comprising, administering to the individual an effective amount of at least one platelet ADP receptor or thienopyridine inhibitor blocking the G coupled PAR-1/PAR-4 on human platelets of the individual, and wherein the ADP-receptor inhibitor or thienopyridine is selected from the group consisting of prasugrel, a prasugrel metabolite, clopidogrel, a clopidogrel metabolite, ticlopidine, a ticlopidine metabolite, cangrelor, a cangrelor metabolite, AZD-6140, and an AZD-6140 metabolite.

The present invention also relates to a method of preventing and/or treating an individual at risk for a vascular event, by blocking expression of platelet PAR-1/PAR-4 receptor of the individual comprising administering to the individual an effective amount of an ADP-receptor inhibitor or a thienopyridine selected from the group consisting of prasugrel, a prasugrel metabolite, clopidogrel, a clopidogrel metabolite, ticlopidine, a ticlopidine metabolite, cangrelor, a cangrelor metabolite, AZD-6140, and an AZD-6140 metabolite.

The present invention also relates to a method of treating or preventing a vascular event, disease or disorder selected from a group consisting of myocardial infarction, angina, pulmonary embolism, transient ischemic attack, deep vein thrombosis, thrombotic re-occlusion subsequent to a coronary intervention procedure, heart surgery or vascular surgery, peripheral vascular thrombosis, Syndrome X, heart failure, and a disorder in which a narrowing of at least one coronary artery occurs caused by, exacerbated by, or related to activation of PAR-1/PAR-4 receptor expression comprising administering to the individual an effective amount of an ADP-receptor inhibitor or thienopyridine selected from the group consisting of prasugrel, a prasugrel metabolite, clopidogrel, a clopidogrel metabolite, ticlopidine, a ticlopidine metabolite, cangrelor, a cangrelor metabolite, AZD-6140, and an AZD-6140 metabolite.

A method of treating an individual with vascular disease comprising administering a therapeutically effective amount of at least one ADP-receptor inhibitor or thienopyridine in a carrier to the individual, wherein the ADP-receptor inhibitor or thienopyridine prevents the over expression of the platelet PAR-1/PAR-4 receptor of the individual, and the ADP-receptor inhibitor or thienopyridine is selected from the group consisting of prasugrel, a prasugrel metabolite, clopidogrel, a clopidogrel metabolite, ticlopidine, a ticlopidine metabolite, cangrelor, a cangrelor metabolite, AZD-6140, and an AZD-6140 metabolite.

The present invention relates to the use of a compound of formula I in combination with other effective antiplatelet and/or anticoagulant agent(s) selected from the group consisting of aspirin, heparin, hirudin, bivalirudin, dipyridamole, statin and a platelet glycoprotein IIb/IIIa inhibitor, angiotensin receptor inhibitor, or selective serotonin reuptake inhibitor for the treatment or prevention of PAR-1/PAR-4 receptor induced coagulation related vascular events.

The present invention relates to the use of 2-acetoxy-5-(□-cyclopropylcarbonyl-2-fluorobenzyl))-4,5,6,7-tetrahydrothieno[3,2-c]pyridine hydrochloric acid addition salt singly or in combination with other effective anti-coagulants for the treatment and/or prevention of PAR-1/PAR-4 receptor induced Vascular Diseases.

### DEFINITIONS

The term, “Vascular Diseases” refers to diseases treatable, preventable, or able to be ameliorated by inhibition of PAR-1/PAR-4 receptor activity (i.e. PAR-1/PAR-4 induced). Examples of Vascular Diseases encompassed by the invention include myocardial infarction, angina, pulmonary embolism, transient ischemic attack, deep vein thrombosis, thrombotic re-occlusion subsequent to a coronary intervention procedure, heart surgery or vascular surgery, peripheral vascular thrombosis, vascular disorders related diabetes mellitus, and/or syndrome X (metabolic suyndrome), heart failure, and a disorder in which a narrowing of at least one coronary artery occurs.

The term “administering” as used herein is intended to include various routes of administration, particularly oral, which allow for a compound (s) of the invention to perform its intended function of treating and/or preventing the occurrence or recurrence of vascular diseases.

The term “treatment” as used herein refers to the amelioration, inhibition, prevention of occurrence or recurrence, reduction in severity or effect of vascular diseases including myocardial infarction, angina, pulmonary embolism, transient ischemic attack, deep vein thrombosis, thrombotic re-occlusion subsequent to a coronary intervention procedure, heart surgery or vascular surgery, peripheral vascular thrombosis, vascular disorders related diabetes mellitus, and/or syndrome X (metabolic syndrome), heart failure, vascular diseases associated with diabetes, and a disorder in which a narrowing of at least one coronary artery occurs.

The term “effective amount” as used herein refers to the amount of a compound of formula I and/or other vascular protective agent (drug) necessary or sufficient to treat or prevent the particular vascular disease in a treatment regimen comprised of a compound of formula I and/or other compounds of the invention including clopidogrel, a clopidogrel metabolite, ticlopidine, a ticlopidine metabolite, cangrelor, a cangrelor metabolite, AZD-6140, and an AZD-6140 metabolite as prescribed by a qualified physician.

The effective amount may vary depending on factors known to one of skill in the art, including for example, the optional combination of compound I with aspirin, the use of drug coated stents, mode and regimen of administration, the size of the subject, genetic determinants of absorption or metabolism, genetic or behavioral predisposition to vascular diseases or the severity and/or potential for recurrence thereof. One of skill in the art would be able to consider these and related factors to make the appropriate determination regarding effective amount.

The phrase “pharmaceutically acceptable carrier” refers to any substance co-administered with the compound of the invention including compound 1 singly and/or in combination with other compounds of the invention and which allows the compound(s) to perform its intended function. Examples of such carriers include solutions, solvents, dispersion media, delay agents, emulsions, microparticles and the like for combination therapies.

As used herein, the terms “thienopyridine therapy” and “treatment with a thienopyridine” are synonymous and mean the use of an approved drug having a thienopyridine core or portion for the treatment, prevention and/or amelioration of diseases caused, related to, or exacerbated by PAR-1/PAR-4 receptor activity.

The phrase “combination therapy,” for the purpose of this invention means the use of a compound of formula I and other effective ADP receptor inhibitors, thienopyridines or anticoagulant selected from the group consisting of low-molecular weight heparins, fondaparinux, direct thrombin inhibitors [including ximelegatran], factor Xa inhibitors, clopidogrel, a clopidogrel metabolite, ticlopidine, a ticlopidine metabolite, cangrelor, a cangrelor metabolite, AZD-6140, and an AZD-6140 metabolite, wherein both treatments are initiated simultaneously or within a short period (typically within 1 to 30 days) after initiation of the first therapy. The phrase also connotes the use of a combination delivery method wherein both chosen therapies are delivered in a single tablet, capsule, inhalation mechanism, intravenous solution or rectal suppository. The period of combination therapy as defined above may be from about 30 days to about 700 days and preferably from about 30 days to about 365 days Ultimately, the precise period of therapy according to this invention is a determination to be made by the treating or attending physician and tailored to the particular patient including considerations of whether the need is acute or chronic.

One aspect of the present invention is in the provision of a compound that reduces the thrombin generation state of an individual or patient. Thrombin is a pluripotent serine protease that plays a central role in hemostasis following tissue injury by converting soluble plasma fibrinogen into an insoluble fibrin clot and by promoting platelet aggregation. In addition to these procoagulant effects, thrombin also influences a number of cellular responses that play important roles in subsequent inflammatory and tissue repair processes. Thrombin influences the recruitment and trafficking of inflammatory cells and is a potent mitogen for a number of cell types, including endothelial cells, fibroblasts, and smooth muscle cells. Thrombin also promotes the production and secretion of extracellular matrix proteins and influences connective tissue remodeling processes. There is increasing in vivo evidence that the pro-inflammatory and pro-fibrotic effects of thrombin play an important role in both normal tissue and vascular repair, as well as in a number of pathological conditions associated with acute or persistent activation of the coagulation cascade, including restenosis and neointima formation following vascular injury, atherosclerosis, pulmonary fibrosis, and glomerulonephritis.

Most of the cellular effects elicited by thrombin are mediated via a family of widely expressed G-protein-coupled receptors, termed protease-activated receptors (PARs) that are activated by limited proteolytic cleavage of the N-terminal extracellular domain. The newly generated N terminus acts as a tethered ligand and interacts intramolecularly with the body of the receptor to initiate subsequent cell signaling events (Chambers R C, Leoni P, Blanc-Brude O P, Wembridge D E, Laurent G J. Thrombin is a potent inducer of connective tissue growth factor production via proteolytic activation of protease-activated receptor-1. J Biol Chem. 2000 Nov. 10; 275(45):35584-91).

Although it has long been recognized that catalytically active thrombin is required for stimulation of platelet aggregation (Phillips D R. Thrombin interaction with human platelets: potentiation of thrombin-induced aggregation and release by inactivated thrombin. Thromb Diathesis Haemorrhag. 1974; 32:207-215) increased interest in the effects of thrombin in cellular stimulation resulted from the identification of a seven-transmembrane domain G-protein-coupled receptor/substrate for thrombin (Vu T H, Hung D T, Wheaton V I, Coughlin S R. Molecular cloning of a functional thrombin receptor reveals a novel proteolytic mechanism of receptor activation. Cell. 1991; 64:1057-1066).

When this receptor, PAR-1,4 is proteolytically cleaved, the new amino terminus functions as a tethered ligand to initiate signal transduction, and peptides derived from the new amino terminus, thrombin receptor agonist peptides (TRAPs), function as agonists for the uncleaved receptor. In addition to platelet aggregation and release, PAR-1 mediates several other cellular responses (Pilcher B K, Kim D W, Carnery D H, Tomasek J J. Thrombin stimulates fibroblast-mediated collagen lattice contraction by its proteolytically activated receptor. Exp Cell Res. 1994; 211:368-373; see also Sower L E, Froelich C J, Carney D H, Fenton J W II, Klimpel G R. Thrombin induces IL-6 production in fibroblasts and epithelial cells: evidence for the involvement of the seven-transmembrane domain (STD) receptor for thrombin. J Immunol. 1995; 155:895-901; and Rabiet M, Plantier J, Rival Y, Genoux Y, Lampugnani M, Del Mar E G. Thrombin-induced increase in endothelial permeability is associated with changes in cell-to-cell junction organization. Arterioscler Thromb Vasc Biol. 1996; 16:488-49) It is now known that PAR-1 interacts with anion binding exosite I of thrombin the site of the mutation in Thrombin Quick I, accounting for the low platelet-aggregating activity of this mutant thrombin. The relatively greater activity of Thrombin Quick I in stimulating thromboxane production suggested that anion binding exosite I is not involved in interaction of thrombin with a second, unidentified receptor/substrate. Since it has now been reported that the hexapeptide derived from the new amino terminus of PAR-1 (TRAP-6) can stimulate thromboxane production by human platelets (Kinlough-Rathbone R L, Rand M L, Packham M A. Rabbit and rat platelets do not respond to thrombin receptor peptides that activate human platelets. Blood. 1993; 82:103-106).

The thrombin stimulates thromboxane production through two proteolytically activated receptors on the platelet surface. One pathway, which does not result in maximal thromboxane production, is stimulated and downregulated by TRAP through PAR-1/3. The second pathway, which is inhibited by genistein, apparently requires at least one tyrosine kinase but does not depend on a [Ca²⁺] flux (Henriksen R A, Samokhin G P, Tracy P B. Thrombin-induced thromboxane synthesis by human platelets. Properties of anion binding exosite I-independent receptor. Arterioseler Thromb Vasc Biol. 1997 December; 17(12):3519-26).

Through the activation of PAR-1, thrombin stimulates production of prostacyclin (Weksler, B. B., C. W. Ley, and E. A. Jaffe. Stimulation of endothelial cell prostacyclin production by thrombin, trypsin, and the ionophore A23187. J. Clin. Invest. 62: 923-930, 1978), increase the cytosolic Ca2+ signal, and induce expression of cell surface adhesion proteins, P-selectin and intercellular adhesion molecule-1 (ICAM-1) (Weksler, B. B., C. W. Ley, and E. A. Jaffe. Stimulation of endothelial cell prostacyclin production by thrombin, trypsin, and the ionophore A23187. J. Clin. Invest. 62: 923-930, 1978).

PAR-1 is the major receptor responsible for mediating most of the pro-inflammatory and pro-fibrinolytic effects of thrombin. Once thrombin has interacted with its receptor, it exerts its cellular effects either directly or via the induction and release of secondary mediators, including classical growth factors, pro-inflammatory cytokines, and vasoactive peptides and amines (Sugama, Y., C. Tiruppathi, K. Janakidevi, T. T. Andersen, J. W. Fenton II, and A. B. Malik. Thrombin-induced expression of endothelial P-selectin and intercellular adhesion molecule-1: a mechanism for stabilizing neutrophil adhesion. J. Cell Biol. 119: 935-944, 1992).

Thrombin also binds with high affinity to the platelet glycoprotein Ib (GPIb) that belongs to the leucine-rich repeat family of proteins (Chambers R C, Leoni P, Blanc-Brude O P, Wembridge D E, Laurent G J. Thrombin is a potent inducer of connective tissue growth factor production via proteolytic activation of protease-activated receptor-1. J Biol. Chem. 2000 Nov. 10; 275(45):35584-91). GPIb is a part of GPIb-IX-V system, that mainly involved into process of platelet adhesion. GPIb binds thrombin with high affinity and contributes to platelet activation by the enzyme; however, there is no evidence that thrombin ligation to GpIb per se is able to trigger platelet activation. Results from available studies show that GpIb can function as a cofactor for PAR-1 cleavage and activation in human platelets. In fact, by directly measuring the hydrolysis of PAR-1 on intact platelets, it was possible to evaluate the effect of the inhibition of thrombin binding to GpIb on PAR-1 cleavage. (Buchanan, S. T., and Gay, N. J. (1996) Prog. Biophys. Mol. Biol. 65, 1-44). But, GP Ib-IX alone, lacking V, is capable of nearly complete adhesive function and surface expression, and GPV is present in only half of the Ib-IX complexes (14). The platelets of GP V −/− mice, possessing only the Ib-IX form of the receptor, display nearly normal function, except for their modest, and perhaps entirely unexpected, increase in reactivity to thrombin (De Candia E, Hall S W, Rutella S, Landolfi R, Andrews R K, De Cristofaro R. Binding of thrombin to glycoprotein Ib accelerates the hydrolysis of PAR-1 on intact platelets. J Biol Chem. 2001 Feb. 16; 276(7):4692-8. Epub 2000 November 17). This initial insight, GP V serves as an inhibitor of the platelet response to thrombin (Kahn, M. L., Diacovo, T. G., Bainton, D. F., Lanza, F., Trejo, J. & Coughlin, S. R. (1999) Blood 94, 4112-4121). Key reagents were protease-active thrombin (capable of activating both PARs and Ib-IX) as contrasted to protease-inactive thrombin (activating only Ib-IX/incapable of activating PARs) (Ramakrishnan, V., Reeves, P. S., DeGuzman, F., Deshpande, U., Ministri-Madrid, K., DuBridge, R. B. & Phillips, D. R. (1999) Proc. Natl. Acad. Sci. USA 96, 13336-13341).

### PREFERRED EMBODIMENTS OF THE INVENTION

One embodiment of the present invention is the use of a pharmaceutical composition comprising a therapeutically effective amount of a compound of formula I, or pharmaceutically acceptable salt, solvate, racemate or enantiomer thereof for the treatment and/or prevention of Vascular Diseases and recurrence thereof.

Also preferred is the use of the combination of aspirin and a compound of formula I, or pharmaceutically acceptable salt, solvate, racemate or enantiomer thereof, in combination with one of either clopidogrel, a clopidogrel metabolite, ticlopidine, a ticlopidine metabolite, cangrelor, a cangrelor metabolite, AZD-6140, and an AZD-6140 metabolite, including low-molecular weight heparins, fondaparinux, direct thrombin inhibitors (including ximelegatran), and factor Xa inhibitors for the treatment and/or prevention of Vascular Diseases and recurrence thereof.

More preferred for the purpose of the invention is the use of a compound of formula I, or a pharmaceutically acceptable salt, solvate, racemate or enantiomer thereof, in combination with aspirin for the inhibition of the activity or activation of PAR-1 and/or PAR-4 receptors for the treatment and/or prevention of Vascular Diseases.

A preferred compound for the practice of the invention is 2-Acetoxy-5-(α-cyclopropylcarbonyl-2-fluorobenzyl)-4,5,6,7-tetrahydrothieno[3,2-c]pyridine hydrochloride having the following formula:

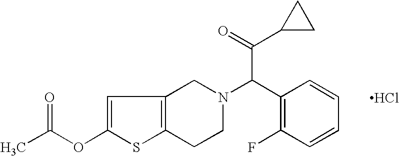

Also preferred for the practice of the invention is the compound 2-Acetoxy-5-(α-cyclopropylcarbonyl-2-fluorobenzyl)-4,5,6,7-tetrahydrothieno[3,2-c]pyridine maleate, which has the following formula:

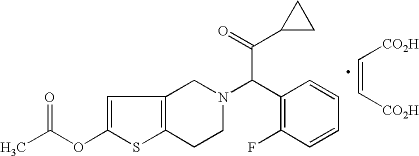

Also preferred for the practice of the invention is the active metabolite of the compound of formula I represented by the structure II:

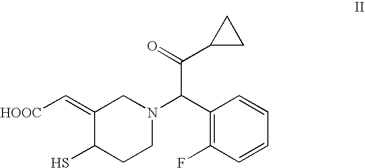

The active metabolite II is a mixture of four enantiomers each of which has shown dose dependent anti-platelet aggregation ability, and thus useful for the practice of the invention. The RS enantiomer has been shown to be most potent and is therefore preferred.

Also preferred for the practice of the invention is a prodrug of the compound of formula I represented by formulae III and IV, their respective pharmaceutically acceptable salts, solvates, racemates or enantiomers thereof:

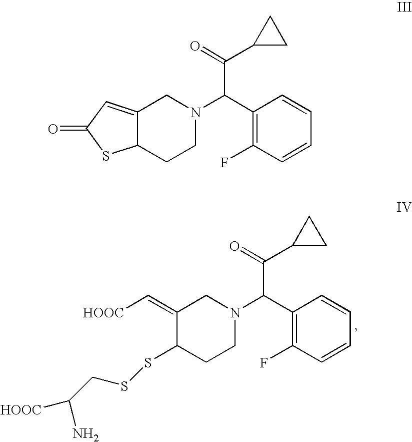

and other conjugates, derivatives or homologs of compound I or II, which may readily cleave to form the active metabolite II. Procedures and processes for making prodrugs described herein are known to one of skill in the art or may be arrived at with minimal experimentation or modifications from those procedures known to one of skill in the art.

The compound clopidogrel is represented by the structure

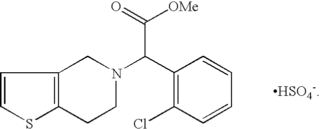

The compound AZD6140 is represented by the structure

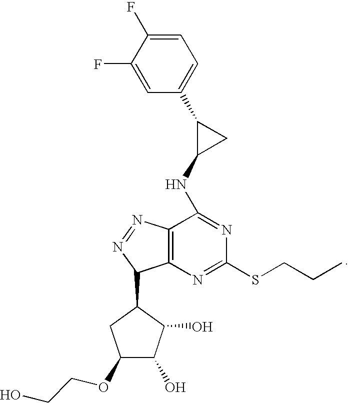

The compound cangrelor from the Medicines Company is represented by the structure

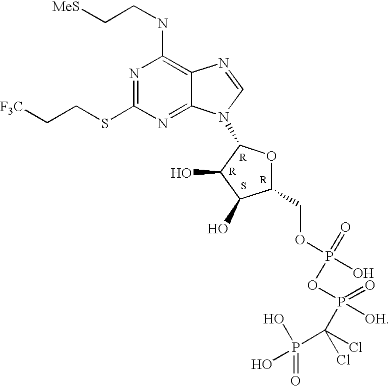

### Preparing Compounds of the Invention

A 2-acetoxy-5-(α-cyclopropylcarbonyl-2-fluorobenzyl)-4,5,6,7-tetrahydrothieno[3,2-c]pyridine or the acid addition salt has an asymmetric carbon in their molecule and in each compound two isomers having R and S configuration can exist. The present invention encompasses an individual isomer or a mixture of these isomers in optional proportions. An optically active isomer of 2-acetoxy-5-(α-cyclopropylcarbonyl-2-fluorobenzyl)-4,5,6,7-tetrahydrothieno[3,2-c]pyridine acid addition salt is prepared using an optically active starting material or is isolated from a racemic mixture of synthetically prepared 2-acetoxy-5-(α-cyclopropylcarbonyl-2-fluorobenzyl)-4,5,6,7-tetrahydrothieno[3,2-c]pyridine acid addition salt by a conventional optical resolution.

Under certain conditions when a (2-acetoxy-5-(α-cyclopropylcarbonyl-2-fluorobenzyl)-4,5,6,7-tetrahydrothieno[3,2-c]pyridine acid addition salt is allowed to stand in contact with the atmosphere or is recrystallized, it may absorb water or may take up water to form a hydrate. The present invention encompasses these hydrates.

The compound of formula I may be prepared by a variety of methods, particularly those disclosed in U.S. Pat. No. 5,288,726, the entire content of which is incorporated herein by reference. The acid addition salts of the compound of formula I may be prepared following procedures disclosed in PCT application WO 02/04461, published Jan. 17, 2002, now U.S. Pat. No. 6,693,115.

Procedures and processes for making other compounds of the invention are known to one of skill in the art based on publications including but not limited to those cited herein and which are hereby incorporated by reference. For example, procedures for preparing clopidogrel are discloseded or may be adduced by one of skill in the art from disclosures in U.S. Pat. Nos. 4,529,596, 4,847,265, 5,576,328, and 6,429,210B1 all of which are incorporated herein by reference. Procedures for preparing Cangrelor and/or related analogs are disclosed in one or more of PCT international publications WO 94/18216, WO 92/17488, WO 98/28009, WO 99/02542, WO 01/39781, and related publications and/or references therein.

Procedures for preparaing AZD6140 and/or related analogs are disclosed in one or more of PCT international publications WO 00/034283, WO 01/092262, WO 01/092263 and related publications and/or references therein.

### Method of Using the Invention

During a substudy of samples from a platelet inhibition study comparing the compound of formula I (prasugrel) versus clopidogrel in patients undergoing coronary stenting, the inventors observed that prasugrel in particular and ADP inhibitors including clopidegrel have beneficial effects on PAR-1 and PAR-4 receptor activities. The study sought to compare the antiplatelet properties of prasugrel and clopidogrel in the frame of a Phase 2 randomized trial.

Specifically, nine patients undergoing coronary stenting were randomized to one of three arms of prasugrel (40-60-60 mg loading, and 7.5-10-15 mg maintenance daily dose), or clopidogrel (300 mg/75 mg). Aspirin and GP IIb/IIIa inhibitors were allowed. Platelet activity was assessed at baseline, at 4, and 24 hours, and at 30 days after stent implantation and samples taken were analyzed for various markers of vascular disease including PAR1/Par-4 inhibition. Results are shown in table I.

TABLE 1
Results (Flow Cytometry Data)

| | Study | | | | |
|---|---|---|---|---|---|
| Parameter | group | Baseline | 4 hr | 24 hr | 30 days |
| CD31 (PECAM- | prasugrel | 69.9 ± 19.3 | 52.5 ± 29.3 | 52.8 ± 28.7 | 24.6 ± 24.4 |
| 1) | clopidogrel | 62.9 ± 12.2 | 41.6 ± 17.3 | 38.0 ± 18.9 | 58.6 ± 8.4 |
| CD42 (GP-Ib) | prasugrel | 145.9 ± 35.5 | 138.7 ± 38.3 | 154.0 ± 39.6 | 110.0 ± 47.7 |
| | clopidogrel | 135.2 ± 35.1 | 134.2 ± 37.3 | 129.8 ± 37.6 | 168.0 ± 48.0 |
| CD62p | prasugrel | 10.8 ± 4.1 | 8.9 ± 3.3 | 7.9 ± 1.4 | 5.2 ± 2.3 |
| (P-selectin) | clopidogrel | 10.4 ± 3.4 | 10.0 ± 3.5 | 9.8 ± 3.6 | 9.6 ± 1.4 |
| CD151+CD14 | prasugrel | 73.0 ± 11.1 | 60.1 ± 18.9 | 62.7 ± 29.2 | 61.8 ± 23.0 |
| | clopidogrel | 67.1 ± 14.5 | 62.6 ± 14.4 | 69.5 ± 22.4 | 66.0 ± 9.5 |
| CD40 ligand | prasugrel | 6.9 ± 0.8 | 4.8 ± 1.4 | 4.6 ± 1.1 | 3.7 ± 1.3 |
| | clopidogrel | 5.5 ± 1.3 | 4.1 ± 1.3 | 4.2 ± 1.3 | 6.5 ± 1.6 |
| CD165 (GP37) | prasugrel | 24.7 ± 2.8 | 17.8 ± 5.6 | 15.8 ± 5.7 | 12.2 ± 2.8 |
| | clopidogrel | 23.9 ± 5.2 | 17.8 ± 5.8 | 18.3 ± 4.2 | 19.7 ± 3.0 |
| WEDE15 | prasugrel | 37.0 ± 9.8 | 17.0 ± 6.2 | 19.6 ± 8.4 | 15.3 ± 5.9 |
| (PAR-1/PAR-4) | clopidogrel | 30.2 ± 10.9 | 20.9 ± 9.3 | 25.7 ± 9.6 | 16.5 ± 3.2 |
| SPAN12 | prasugrel | 19.5 ± 5.4 | 13.3 ± 2.5 | 12.0 ± 2.7 | 6.6 ± 1.7 |
| (PAR-1/PAR-4) | clopidogrel | 18.0 ± 4.8 | 16.2 ± 5.0 | 17.1 ± 5.3 | 6.2 ± 0.9 |
| Thrombospondin | prasugrel | 7.3 ± 2.2 | 6.4 ± 1.1 | 6.7 ± 1.7 | 4.0 ± 0.7 |
| | clopidogrel | 7.1 ± 2.0 | 6.7 ± 2.0 | 7.8 ± 2.8 | 8.1 ± 1.3 |

Independent of the dosing regimen, patients treated with prasugrel exhibited more potent platelet inhibition as determined by ADP-, and collagen-induced aggregation, Ultegra® Analyzer, and surface expression of PECAM-1, GPIIb/IIIa antigen, and activity with PAC-1 antibody, GPIb, P-selectin, CD40-ligand, GP37, and thrombospondin receptor when compared with those treated with clopidogrel, hence the particular preference for the use of Prasugrel in the practice of the present invention. Applicants believe the above results are significant and establish a correlation between use of CS-747 (prasugrel) and inhibition of the activity or activation of PAR-1 and/or PAR-4 receptors. To a lesser degree, a similar correlation is established between use of Clopidogrel, the active ingredient in Plavix/Iscover, and PAR-1/PAR-4 expression. The correlations or trends shown in table 1 are independent of aspirin or Gb-11b inhibitor use by some patients at baseline. Confounding effects by aspirin or GB-IIb/IIIA use at baseline should be absent at 30 days.

The advantages to be obtained by the use of a compound of formula I, other compound(s) of the invention or a combination of compounds of the invention include simultaneous platelet aggregation inhibition and inhibition of PAR-1/PAR-4 receptor expression. Thus, the invention provides a compound of formula I (Prasugrel) or other compound of the invention disclosed herein singly or in combination with other ADP inhibitors as a potent dual anticoagulant and anti thrombotic agent. Moreover, additional advantage is to be gained by the ability of Prasugrel to be delivered orally either as an antiplatelt agent, an anticoagulant or for patients where it is indicated, as both an antiplatelet agent and an anticoagulant.

The combination of a compound of formula I, or other compound of the invention and aspirin for the purpose of practicing the invention may be accomplished by having individual or unit doses of the compound of formula I or other compound of the invention and aspirin or by having a combined prepackaged or pre-formulated dose of aspirin and the compound of formula I or other compound of the invention.

The specific dose of a compound of the present invention administered to obtain therapeutic or prophylactic effect will, of course, be determined by the particular circumstances of the patient, including, for example, the route of administration and the particular Vascular Disease being treated. Typical doses will contain a non-toxic dosage level of from about 0.01 mg/kg to about 50 mg/kg of body weight of a compound of the invention. More preferred doses of the compound(s) of the invention singly or in combination as tablets or capsules contain from 5 mg to 100 mg of Active Ingredient per dose to an average weight patient or calibrated for the patient's weight and health characteristics. The frequency of dosing and length of dosing are determinations to be made by the treating physician(s) to achieve maximum efficacy for the particular patient and circumstance.

The co-administration of aspirin in combination or conjunction with as compound or compounds of the invention to obtain therapeutic or prophylactic effect will of course be determined by the particular circumstances of the patient. In general the amount of aspirin for the purpose of the present invention is about that generally approved for the particular patient population, e.g. from about 75 mg to about 300 mg of aspirin 1 to 3 times daily.

For the pharmaceutical formulations of compound I with or without aspirin or other compound of the invention, any suitable carrier known to one of skill in the art may be used. In such a formulation, the carrier may be a solid, liquid, or mixture of a solid and a liquid. For example, the Active Ingredient may be dissolved in a suitable solvent at a concentration of about 2 to 200 mg/mL in a 4% dextrose/0.5% Na citrate aqueous solution. Solid form formulations for impregnation on the stent include powders and pastes. A solid carrier can be one or more substance, which may also act as lubricants, solubilizers, suspending agents, and pharmaceutically acceptable adhesive agents.

In powders, the carrier is a finely divided solid having the necessary binding properties in suitable proportions, which is in an admixture with the finely divided Active Ingredient. The powders will typically be sprayed on optionally followed by spray-on of annealing or sealing agents. The powders preferably contain from about 1 to about 99 weight percent of the Active Ingredient. Suitable solid carriers are magnesium carbonate, magnesium stearate, talc, sugar lactose, pectin, dextrin, starch, gelatin, tragacanth, methylcellulose, sodium carboxymethyl cellulose, pharmaceutically acceptable low melting waxes, and pharmaceutically acceptable adhesives.

The Active Ingredient may be dissolved or suspended in a pharmaceutically acceptable carrier, such as sterile water, sterile organic solvent or a mixture of both. The Active ingredient may also be dissolved in a suitable organic solvent, for instance aqueous propylene glycol. Dispersing the finely divided Active ingredient in aqueous starch or sodium carboxymethyl cellulose solution, or binder or pharmaceutically acceptable adhesive may result in other compositions. Impregnate the solution or suspension on a stent by coating the admixture of active ingredient on the stent and allowing the solvent to evaporate slowly under vacuum until nearly all solvent or liquid is evaporated.

The following pharmaceutical formulations are illustrative only and are not intended to limit the scope of the invention in any way. “Active ingredient”, refers to a compound according to Formula (I) and/or other compound of the invention optionally including aspirin which is/are to be administered to a patient in need thereof.

### Slow Release Formulation 1.

Hard gelatin powder is prepared using the following ingredients:

| | Quantity | |
|---|---|---|
| | (mg/capsule) | |
| Active ingredient | 20 | |
| Starch, dried | 200 | |
| Magnesium stearate | 10 | |
| Total | 230 | mg |

### Formulation 2

A solid composition of formula I is prepared using the ingredients below:

| | Quantity | |
|---|---|---|
| | (mg/tablet) | |
| Active ingredient | 5 | |
| Cellulose, microcrystalline | 400 | |
| Silicon dioxide, fumed | 10 | |
| Stearic acid | 5 | |
| Total | 420 | mg |

The components are blended and compressed to form a solid each weighing 425 mg which is then tableted or capsuled or admixed with a pharmaceutically acceptable adhesion agent.

### Formulation 3

A solid composition of formula I is prepared using the ingredients below:

| | Quantity | |
|---|---|---|
| | (mg/tablet) | |
| Active ingredient | 10 | |
| Cellulose, microcrystalline | 400 | |
| Silicon dioxide, fumed | 10 | |
| Stearic acid | 5 | |
| Total | 425 | mg |

The components are blended and compressed to form a solid each weighing 425 mg that is then tableted or capsuled or admixed with a pharmaceutically acceptable adhesion agent.

### Formulation 4

A solid composition of formula I is prepared using the ingredients below:

| | Quantity | |
|---|---|---|
| | (mg/tablet) | |
| Active ingredient | 20 | |
| Cellulose, microcrystalline | 400 | |
| Silicon dioxide, fumed | 10 | |
| Stearic acid | 5 | |
| Total | 435 | mg |

The components are blended and compressed to form a solid each weighing 425 mg. The solid is then tableted or capsuled or admixed with a pharmaceutically acceptable adhesion agent.

1. Use of a compound of formula I 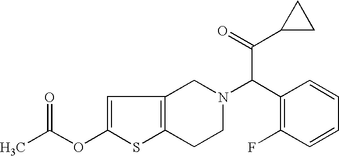
for the treatment of and/or prevention of coagulation induced vascular diseases and recurrence thereof, in a patient in need thereof.

2. Use of PAR-1/PAR-4 inhibitors selected from the group consisting of prasugrel, prasugrel metabolite, clopidogrel, a clopidogrel metabolite, ticlopidine, a ticlopidine metabolite, cangrelor, a cangrelor metabolite, AZD-6140, and an AZD-6140 metabolite for the treatment of coagulation induced vascular diseases.

3. Use according to claim 1 wherein prasugrel is administered orally, by injection, intravenously, intramuscularly, subcutaneously, parenterally, nasally, by inhalation, or by implant.

4. A method of treating an individual at risk for a vascular event, disease or disorder, comprising administering to the individual an effective amount of a platelet ADP-receptor inhibitor, or thienopyridine wherein the ADP-receptor inhibitor, or thienopyridine inhibits the expression of platelet PAR-1/PAR-4 receptor of the individual, and the ADP-receptor inhibitor is selected from the group consisting of prasugrel, a prasugrel metabolite, clopidogrel, a clopidogrel metabolite, ticlopidine, a ticlopidine metabolite, cangrelor, a cangrelor metabolite, AZD-6140, and an AZD-6140 metabolite.

5. The method of claim 4, wherein the vascular event, disease or disorder is selected from a group consisting of: myocardial infarction, angina, pulmonary embolism, transient ischemic attack, deep vein thrombosis, thrombotic re-occlusion subsequent to a coronary intervention procedure, heart surgery or vascular surgery, peripheral vascular thrombosis, vascular diseases associated with diabetes mellitus, and/or Syndrome X (metabolic syndrome), heart failure, vascular complications of diabetes, and a disorder in which a narrowing of at least one coronary artery occurs.

6. The method of claim 4, wherein the ADP-receptor inhibitor or thienopyridine is administered orally, by injection, intravenously, intramuscularly, subcutaneously, parenterally, nasally, by inhalation, by implant, or by suppository.

7. (canceled)
